(19) United States
(12) Patent Application Publication
(10) Pub. No.: US 2021/0393862 A1
(43) Pub. Date: Dec. 23, 2021

(54) DIALYSIS SOLUTION PRODUCTION DEVICE

(71) Applicant: NIHON TRIM CO., LTD., Osaka-shi (JP)

(72) Inventor: Naoki NAKANISHI, Osaka-shi (JP)

(21) Appl. No.: 17/279,894

(22) PCT Filed: Sep. 11, 2019
(86) PCT No.: PCT/JP2019/035773

§ 371 Date: Mar. 25, 2021

(30) Foreign Application Priority Data
Oct. 1, 2018 (JP) 2018-186859

(51) Int. Cl. A61M 1/16

(52) U.S. Cl. CPC A61M 1/1656; A61M 2202/02; A61M 2205/3334

(57) ABSTRACT

The apparatus (1) for manufacturing dialysate includes a reverse osmosis membrane treatment device (9), a dialysate supply device (26), a dialysis device (40) supplied with the dialysate (27) from the dialysate supply device (26), and a hydrogen dissolution device (6) interposed between the dialysate supply device (26) and the dialysis device (40) and configured to dissolve hydrogen gas in the dialysate (27). The hydrogen dissolution device (6) includes an air supply module (7) connected to the dialysate supply device (26) and configured to dissolve hydrogen gas in the dialysate (27), and a hydrogen supply device (8) connected to the air supply module (7) and configured to supply hydrogen gas to the air supply module (7).

### CROSS REFERENCE TO RELATED APPLICATIONS

This application is a national stage application, filed under 35 U.S.C. § 371, of International Application No. PCT/JP2019/035773, filed Sep. 11, 2019, which international application claims priority to and the benefit of Japanese Application No. 2018-186859, filed Oct. 1, 2018; the contents of both which as are hereby incorporated by reference in their entireties.

### BACKGROUND

### Technical Field

The present invention relates to an apparatus for manufacturing dialysate.

### Description of Related Art

Hemodialysis has been known as one of the effective treatments for patients with renal failure who have decreased renal function and are unable to excrete urine to regulate water content and remove harmful substances inside the body including waste products such as urea.

Such hemodialysis is a treatment method in which the following operation is repeatedly performed: Blood is drawn out of a body using a blood pump, and the dialysate and the blood are brought into contact with each other via a dialyzer, whereby a diffusion phenomenon due to a concentration gradient is utilized to remove harmful substances in the body and water from the blood, and then the blood is returned to the body again (re-transfusion).

Further, in recent years, it has been observed that oxidative stress occurs in dialysis patients in the hemodialysis. It is considered that the oxidative stress is caused by active oxygen generated during dialysis, and it has been proposed to eliminate this active oxygen to reduce the oxidative stress.

For example, an apparatus for manufacturing dialysate is disclosed according to which dialysate is prepared by dissolving hydrogen gas in dialysate source reagent (agent A containing glucose, sodium, and the like) and then mixing the dialysate source reagent with another dialysate source reagent (agent B containing bicarbonate). It is disclosed that such configuration makes it possible to manufacture dialysate with lowered oxidation-reduction potential (see, for example, Japanese Patent No. 5872321).

### BRIEF SUMMARY

However, in the apparatus for manufacturing according to Japanese Patent No. 5872321, there has been known the following problem: In preparing the dialysate, the dialysate source reagent containing hydrogen gas is mixed with the other dialysate source reagent and then diffused, resulting in that the concentration of dissolved hydrogen in acral dialysate (i.e., dialysate supplied to a dialysis device and used for purification of patient's blood through dialyzer) decreases.

Therefore, the present invention has been made in view of the above problems, and an object of the present invention is to provide an apparatus for manufacturing dialysate, which can effectively reduce a decrease in the concentration of dissolved hydrogen in acral dialysate.

In order to achieve the above objective, the apparatus for manufacturing dialysate of the present invention includes: a reverse osmosis membrane treatment device configured to perform reverse osmosis membrane treatment to water; a dialysate supply device connected to the reverse osmosis membrane treatment device, and configured to prepare and supply dialysate obtained through mixing reverse osmosis water treated by the reverse osmosis membrane treatment device with undiluted dialysate; and a dialysis device connected to the dialysate supply device and configured to be supplied with the dialysate from the dialysate supply device, in which a hydrogen dissolution device configured to dissolve hydrogen gas in the dialysate and is interposed between the dialysate supply device and the dialysis device, and the hydrogen dissolution device is connected to the dialysate supply device and the dialysis device, and includes: an air supply module configured to dissolve hydrogen gas in the dialysate; and a hydrogen supply device connected to the air supply module and configured to supply hydrogen gas to the air supply module.

According to the present invention, a decrease in the concentration of dissolved hydrogen in acral dialysate can be effectively reduced.

### BRIEF DESCRIPTION OF THE FIGURES

FIG. 1 is a schematic view showing a configuration of an apparatus for manufacturing dialysate according to a first embodiment of the present invention.

FIG. 2 is a schematic view showing a configuration of an apparatus for manufacturing dialysate according to a second embodiment of the present invention.

FIG. 3 is a flowchart for explaining a method of adjusting the concentration of dissolved hydrogen by the hydrogen dissolution device according to the second embodiment of the present invention.

FIG. 4 is a schematic diagram of a configuration of an apparatus for manufacturing dialysate according to a variation of the present invention.

### DETAILED DESCRIPTION OF VARIOUS EMBODIMENTS

### First Embodiment

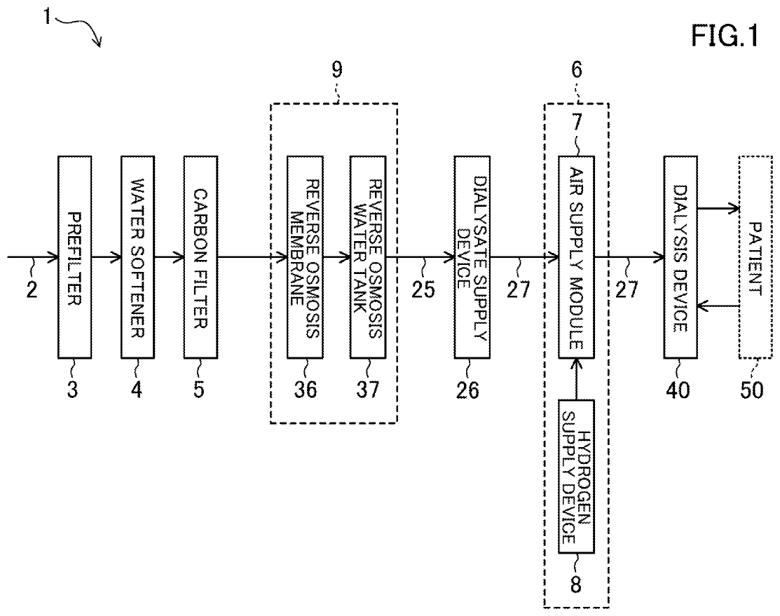

FIG. 1 is a schematic view showing a configuration of an apparatus for manufacturing dialysate according to a first embodiment of the present invention.

The apparatus 1 for manufacturing dialysate includes a prefilter 3, a water softener 4 connected to the prefilter 3, a carbon filter (active carbon treatment device) 5 connected to the water softener 4, a reverse osmosis membrane treatment device 9 connected to the carbon filter 5, and a dialysate supply device 26 connected to the reverse osmosis membrane treatment device 9.

The prefilter 3 removes impurities (for example, iron rust, sand particles, etc.) from raw water 2 (hard water containing dissolved solids such as calcium ions and magnesium ions as hardness components).

The water softener 4 performs demineralization treatment by removing hardness components from the raw water 2 through substitutional reaction due to ion exchange. Note that the raw water 2 in the present embodiment may be tap water, well water, or groundwater.

The carbon filter 5 performs treatment for removing residual chlorine, chloramine, organic substances, and the like contained in the raw water that has been treated by the water softener 4 through physical adsorption action using active carbon which is a porous adsorption material.

As the water softener 4 and the carbon filter 5, known devices may be used.

The reverse osmosis membrane treatment device 9 performs the following treatment (reverse osmosis treatment): When solutions with different concentrations coexist with a semipermeable membrane interposed therebetween, against a phenomenon in which water moves from a solution with lower concentration to a solution with higher concentration (i.e., osmosis), pressure is applied to the solution with higher concentration so that water move from the solution with higher concentration to the solution with lower concentration, thereby obtaining water osmosed into the solution with lower concentration.

Since impurities such as trace metals can be further removed from the raw water obtained by the above series of treatments using the reverse osmosis membrane treatment device 9, it is possible to obtain water (reverse osmosis water) that satisfies the water quality standard specified in ISO13959 (water standard for dialysis).

As shown in FIG. 1, the reverse osmosis membrane treatment device 9 includes a reverse osmosis membrane 36 that performs the above reverse osmosis membrane treatment to the raw water that has been subjected to a treatment performed by the carbon filter 5, and a reverse osmosis water tank 37 for storing the reverse osmosis water that has been subjected to the reverse osmosis membrane treatment.

Further, as shown in FIG. 1, a dialysate supply device 26 is connected to the reverse osmosis membrane treatment device 9. The reverse osmosis water 25 that has been subjected to the treatment performed by the reverse osmosis membrane treatment device 9 is supplied to the dialysate supply device 26.

In the dialysate supply device 26, the dialysate 27 is prepared by mixing the reverse osmosis water 25 supplied and undiluted dialysate. The dialysate 27 thus obtained is supplied to the dialysis device 40 connected to the dialysate supply device 26 to purify the blood of the patient 50. That is, the dialysate supply device 26 also functions as a device that supplies the dialysate 27 prepared to the dialysis device 40.

The undiluted dialysate may be used alone, or two or more kinds of undiluted dialysate may be used in combination.

The dialysis device 40 includes a dialyzer (not shown) that is a blood purification device for purifying blood. When hemodialysis is performed, the blood of the patient 50 is first sent to the dialyzer. Then, the purification of blood is performed by the dialyzer. Thereafter, the blood that has been purified by the dialyzer returns to the body of the patient 50.

As described above, there has been known a problem in conventional apparatuses for manufacturing dialysate that the concentration of dissolved hydrogen in acral dialysate (dialysate supplied to the dialysis device and for purifying the blood of the patient through the dialyzer) decreases.

In view of this, in the present embodiment, the dialysate supply device 26 is configured such that hydrogen gas is dissolved in the dialysate 27 that has been prepared.

More specifically, as shown in FIG. 1, in the apparatus 1 for manufacturing dialysate of the present embodiment, the hydrogen dissolution device 6 for dissolving hydrogen gas in the dialysate 27 is interposed between the dialysate supply device 26 and the dialysis device 40.

The hydrogen dissolution device 6 includes an air supply module 7 that is connected to the dialysate supply device 26 and the dialysis device 40 and dissolves hydrogen gas in the dialysate 27, and a hydrogen supply device 8 that is connected to the air supply module 7 and supplies hydrogen gas to the air supply module 7.

The air supply module 7 includes a plurality of hollow fibers connected to the dialysate supply device 26 and the dialysis device 40. The dialysate 27 that has been prepared by the dialysate supply device 26 is supplied into the inside of the hollow fibers. Further, the hydrogen supply device 8 supplies hydrogen gas to the outside of the hollow fibers. Numerous minute pores are formed in the hollow fibers such that the pores pass through the outer and inner circumferences of the hollow fibers. The walls of the hollow fibers are made of semipermeable membranes. Hydrogen gas permeates into the dialysate 27 through these semipermeable membranes. In the present embodiment, with such a configuration, the dialysate 27 with hydrogen gas dissolved therein is manufactured.

Then, hydrogen gas is supplied to the dialysate 27 while hydrogen gas flows through the outside of the hollow fibers with dialysate 27 supplied to the inside of the hollow fibers.

In addition, by using such hollow fibers, only hydrogen gas is dissolved in the dialysate 27. Thus, it is possible to reduce a disadvantageous problem that bacteria or the like are mixed into the dialysate 27 to contaminate the dialysate 27.

The hydrogen supply device 8 is not particularly limited as long as it can supply hydrogen gas to the air supply module 7. For example, a hydrogen generator that electrolyzes water, a hydrogen generating agent such as magnesium hydride that reacts with water to generate hydrogen, a hydrogen gas cylinder, and the like can be used.

As described above, in the present embodiment, the hydrogen dissolution device 6 for dissolving hydrogen gas in the dialysate 27 is interposed between the dialysate supply device 26 and the dialysis device 40.

Hence, after preparation of the dialysate 27, it is possible to dissolve hydrogen gas in the dialysate 27. Accordingly, a decrease in the concentration of dissolved hydrogen in acral dialysate 27 can be effectively reduced.

The hydrogen dissolution device 6 only includes the air supply module 7 that dissolves hydrogen gas in the dialysate 27 and the hydrogen supply device 8 that supplies hydrogen gas to the air supply module 7. Hence, it is possible to supply hydrogen gas to the dialysate 27 with a simple configuration.

Further, the air supply module 7 with a plurality of hollow fibers is used to bring hydrogen gas into contact with the dialysate 27 to dissolve the hydrogen gas. Thus, hydrogen gas can be effectively supplied.

### Second Embodiment

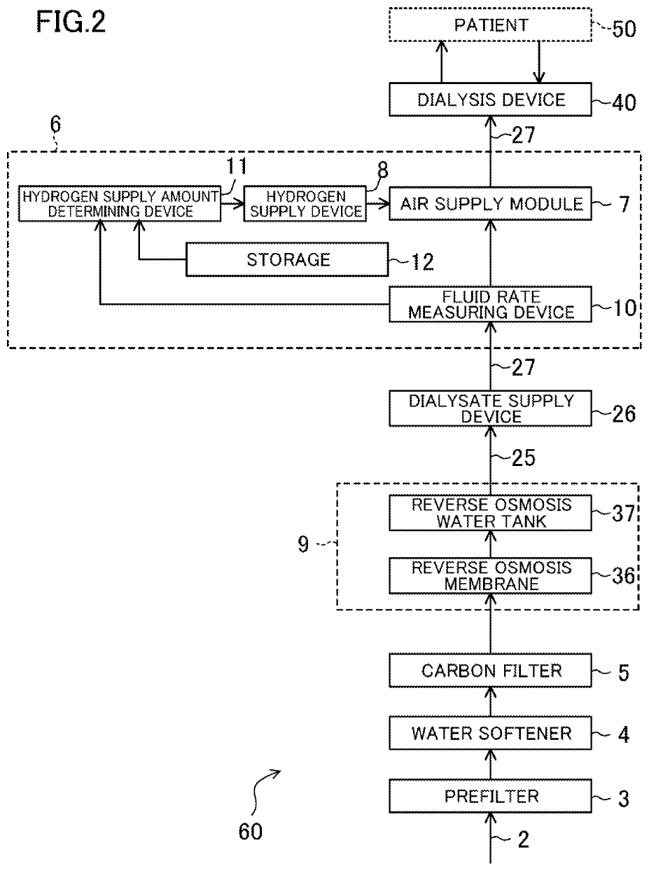

Next, a second embodiment will be described. FIG. 2 is a view showing an apparatus for manufacturing dialysate according to the second embodiment of the present invention. Note that the same reference characters as those in the first embodiment are used to represent equivalent elements, and the detailed explanation thereof will be omitted. The overall configuration of the apparatus for manufacturing dialysate is similar to that of the first embodiment described above, and thus detailed description thereof is omitted here.

In the apparatus 60 for manufacturing dialysate of the present embodiment, the hydrogen dissolution device 6 determines the amount of hydrogen to be supplied corresponding to a fluid rate (flowing fluid rate per unit time) of the dialysate 27 to be supplied to the dialysis device 40 and supplies hydrogen gas to the air supply module 7 to maintain the concentration of dissolved hydrogen in the dialysate 27 at a desired concentration.

Specifically, as shown in FIG. 2, the hydrogen dissolution device 6 according to the present embodiment includes a fluid rate measuring device 10 that is arranged on a water inflow side of the hydrogen dissolution device 6 and measures the fluid rate of the dialysate 27 supplied from the dialysate supply device 26 to the dialysis device 40, and a hydrogen supply amount determining device 11 that is connected to the fluid rate measuring device 10 and determines the amount of hydrogen supplied to the hydrogen supply device 8. Further, the hydrogen dissolution device 6 is connected to the hydrogen supply amount determining device 11 and includes a storage 12 storing data of a target value of the amount of hydrogen supply corresponding to the fluid rate of the dialysate 27.

The fluid rate measuring device 10 is not particularly limited as long as it can detect the fluid rate of the dialysate 27.

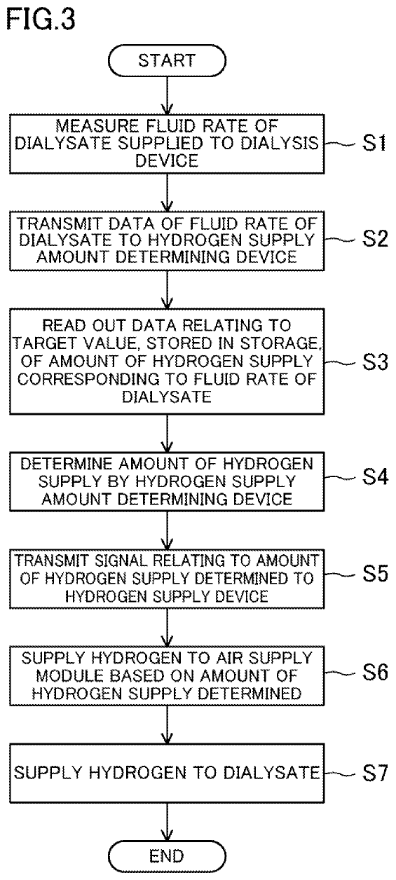

Next, a method of adjusting the concentration of dissolved hydrogen using the hydrogen dissolution device 6 will be described. FIG. 3 is a flowchart for explaining a method of adjusting the concentration of dissolved hydrogen by the hydrogen dissolution device according to the present embodiment.

First, the fluid rate measuring device 10 measures the fluid rate of the dialysate 27 supplied from the dialysate supply device 26 to the dialysis device 40 (step S1).

Then, data of the fluid rate of the dialysate 27 measured by the fluid rate measuring device 10 is transmitted to the hydrogen supply amount determining device 11 (step S2).

Next, the hydrogen supply amount determining device 11 reads out the data stored in the storage 12 on the target value of the amount of hydrogen supply corresponding to the fluid rate of the dialysate 27 (step S3).

After that, the hydrogen supply amount determining device 11 determines the hydrogen supply amount based on the value of the fluid rate of the dialysate 27 measured by the fluid rate measuring device 10 and the target value, stored in the storage 12, of the amount of hydrogen supply corresponding to the fluid rate of the dialysate 27 (step S4).

Subsequently, the hydrogen supply amount determining device 11 transmits, to the hydrogen supply device 8, a signal relating to the determined amount of hydrogen supply (step S5).

Then, the hydrogen supply device 8 supplies hydrogen to the air supply module 7 based on the hydrogen supply amount determined by the hydrogen supply amount determining device 11 (step S6). In the air supply module 7, hydrogen gas is supplied to the dialysate 27 (step S7).

As described above, according to the present embodiment, the hydrogen supply amount determining device 11 determines the amount of hydrogen supply in accordance with the value of the flow rate of the dialysate 27 measured by the fluid rate measuring device 10 and the target value, stored in the storage 12, of the amount of hydrogen supply corresponding to the flow rate of the dialysate 27. The hydrogen supply device 8 supplies hydrogen gas to the air supply module 7 based on the amount of hydrogen supply determined by the hydrogen supply amount determining device 11.

Hence, a decrease in the concentration of dissolved hydrogen in acral dialysate 27 can be effectively reduced and a desired concentration of dissolve hydrogen can be obtained.

The above embodiment may be changed as follows.

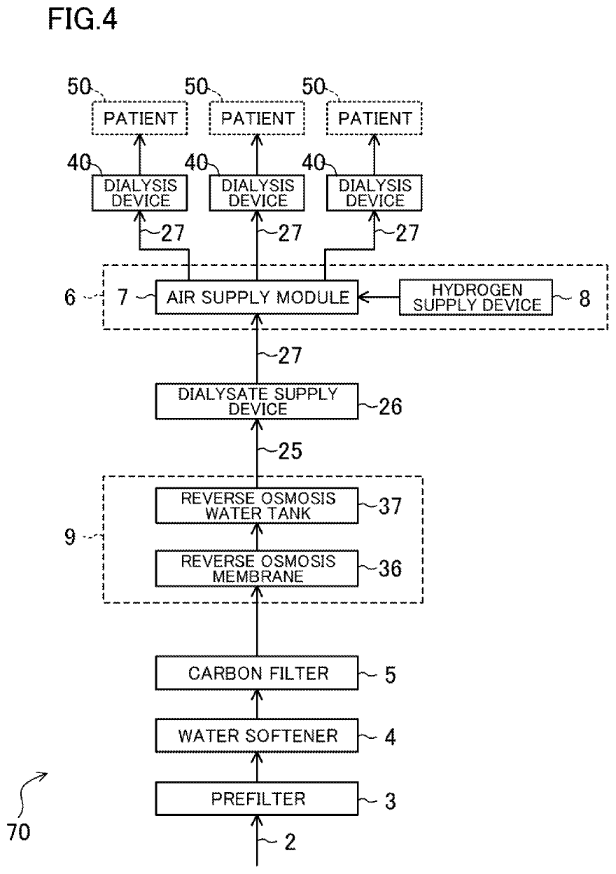

In the above embodiment, a single dialysis device 40 is connected to a single dialysate supply device 26 via the hydrogen dissolution device 6. As is the apparatus 70 for manufacturing dialysate shown in FIG. 4, a plurality of dialysis devices 40 (three in FIG. 4) may be connected to a single dialysate supply device 26 via the hydrogen dissolution device 6.

Accordingly, with a single dialysate supply device 26, it is possible to effectively reduce a decrease of the concentration of dissolved hydrogen in acral dialysate 27 (i.e., dialysate supplied to a plurality of patients 50) in the respective dialysis devices 40, and to obtain a desired concentration of dissolved hydrogen.

As described above, the present invention is particularly useful for an apparatus for manufacturing dialysate with hydrogen gas dissolved therein.

1-5. (canceled)

6. An apparatus for manufacturing dialysate, comprising:
a reverse osmosis membrane treatment device configured to perform reverse osmosis membrane treatment to water;
a dialysate supply device connected to the reverse osmosis membrane treatment device, and configured to prepare and supply dialysate obtained through mixing reverse osmosis water treated by the reverse osmosis membrane treatment device with undiluted dialysate; and
a dialysis device connected to the dialysate supply device and configured to be supplied with the dialysate from the dialysate supply device,
wherein:
a hydrogen dissolution device configured to dissolve hydrogen gas in the dialysate is interposed between the dialysate supply device and the dialysis device, and
the hydrogen dissolution device includes an air supply module that is connected to the dialysate supply device and the dialysis device and configured to dissolve hydrogen gas in the dialysate; and a hydrogen supply device connected to the air supply module and configured to supply hydrogen gas to the air supply module.

7. The apparatus of claim 6, wherein:
the air supply module comprises a plurality of hollow fibers connected to the dialysate supply device and the dialysis device, and
the hydrogen gas is supplied to the dialysate while the hydrogen gas flows through an outside of hollow fibers with the dialysate supplied to an inside of the hollow fibers.

8. The apparatus of claim 6, wherein the hydrogen dissolution device comprises:
a fluid rate measuring device configured to measure a fluid rate of the dialysate supplied to the dialysis device;
a hydrogen supply amount determining device connected to the fluid rate measuring device and the hydrogen supply device and configured to determine an amount of hydrogen supply by the hydrogen supply device; and
a storage connected to the hydrogen supply amount determining device and configured to store data on a target value of the hydrogen supply amount corresponding to the fluid rate of the dialysate,
wherein:
the hydrogen supply amount determining device is configured to determine the amount of hydrogen supply in accordance with a value of the fluid rate of the dialysate measured by the fluid rate measuring device and the target value, stored in the storage, of the amount of hydrogen supply corresponding to the fluid rate of the dialysate, and
the hydrogen supply device is configured to supply the hydrogen gas to the air supply module based on the amount of hydrogen supply determined by the hydrogen supply amount determining device.

9. The apparatus of claim 8, wherein the fluid rate measuring device is a flow rate sensor.

10. The apparatus of claim 6, wherein the dialysis device comprises a plurality of dialysis devices.
